(19) United States
(12) Patent Application Publication
(10) Pub. No.: US 2015/0231198 A1
(43) Pub. Date: Aug. 20, 2015

(54) COMBINATION THERAPY FOR HEMATOLOGICAL MALIGNANCIES

(71) Applicant: Celgene Corporation, Summit, NJ (US)

(72) Inventors: Cristiana CARNITI, Milan (IT); Silvia GIMONDI, Peschiera Borromeo (IT); Antonio VENDRAMIN, Caravaggio (IT); Paolo Corradini, Vedano al Lambro (IT)

(21) Appl. No.: 14/624,487

(22) Filed: Feb. 17, 2015

Related U.S. Application Data
(60) Provisional application No. 61/941,364, filed on Feb. 18, 2014.

(51) Int. Cl. A61K 38/15; A61K 9/00; A61K 31/4184

(52) U.S. Cl. CPC A61K 38/15; A61K 31/4184; A61K 9/0019

(57) ABSTRACT

Methods of treating, preventing or managing T-cell lymphomas are disclosed. The methods encompass administration of an HDAC inhibitor romidepsin and an alkylating agent bendamustine, also known as TREANDA®. Pharmaceutical compositions and single unit dosage forms suitable for use in the methods provided herein are also disclosed

### CROSS REFERENCE TO RELATED APPLICATIONS

This application claims the benefit of priority to U.S. Provisional Patent Application Ser. No. 61/941,364 filed Feb. 18, 2014, the disclosure of which is incorporated by reference herein in its entirety.

### FIELD

Provided are methods for treating hematological malignancies, including T-cell lymphomas, using a combination of a histone deacetylase (HDAC) inhibitor and an alkylating agent. In one embodiment, the HDAC inhibitor is romidepsin. In another embodiment, the alkylating agent is bendamustine. In yet another embodiment, the T-cell lymphoma is peripheral T-cell lymphoma (PTCL).

### BACKGROUND

Lymphoma is a cancer in the lymphatic cells of the immune system. Typically, lymphomas present as a solid tumor of lymphoid cells. These malignant cells often originate in lymph nodes, presenting as an enlargement of the node, i.e., a tumor. It can also affect other organs in which case it is referred to as extranodal lymphoma. Extranodal sites include the skin, brain, bowels and bone. Lymphomas are closely related to lymphoid leukemias, which also originate in lymphocytes but typically involve only circulating blood and the bone marrow cells and do not usually form static tumors (Parham, P. The immune system. New York: Garland Science. p. 414, 2005). Treatment involves chemotherapy and in some cases radiotherapy and/or bone marrow transplantation, and can be curable depending on the histology, type, and stage of the disease (Parham, P., supra).

Classification of lymphomas is complicated. The most accepted by skilled artisan classification defines lymphomas as non-Hodgkin lymphomas (NHLs) (including mature B-cell lymphomas, mature T-cell and natural killer cell lymphomas), Hodgkin's lymphomas and immunodeficiency-associated lymphoproliferative disorders.

Peripheral T-cell lymphomas (PTCLs) are uncommon and aggressive non-Hodgkin lymphomas that develop in mature white blood cells (T cell and natural killer (NK) cells. There may exist in indolent (slow growing) and aggressive (fast growing) form. Aggressive T-cell lymphomas are included within the nodal, extranodal, and leukemic groups.

Peripheral T-cell lymphomas represent approximately 10-15% of all non-Hodgkin lymphomas in the Western world, and their incidence is increasing. Cases of PTCL tend to have an aggressive clinical course, with poor patient responses to conventional chemotherapy and poor long-term survival. So far, treatment approaches include cyclophosphamide, doxorubicin, vincristine, and prednisone and the like chemotherapy although the results are suboptimal.

Romidepsin has been shown to have anticancer activities. The drug is approved in the U.S. for treatment of cutaneous T-cell lymphoma (CTCL) and peripheral T-cell lymphoma (PTCL), and is currently being tested, for example, for use in treating patients with other hematological malignancies (e.g, multiple myeloma, etc.) and solid tumors (e.g., prostate cancer, pancreatic cancer, etc.). It is thought to act by selectively inhibiting deacetylases (e.g., histone deacetylase, tubulin deacetylase), promising new targets for development of a new class of anti-cancer therapies (Bertino & Otterson, Expert Opin Investig Drugs 20(8):11151-1158, 2011). One mode of action involves the inhibition of one or more classes of histone deacetylases (HDAC).

The current success of HDACs and alkylating agents in the clinical practice for PTCL treatment encourages the pursuing of combinational therapy in order to increase the response rate. An effective and safe combinational therapy would be very valuable in a type of cancer where few treatment alternatives exist.

### SUMMARY

In one embodiment, provided herein are methods for treating, preventing or managing hematological malignancies in a patient, comprising administering to said patient an effective amount of an HDAC inhibitor in combination with an alkylating agent.

HDAC inhibitors useful in the methods provided herein include, but are not limited to, trichostatin A (TSA), Vorinostat (SAHA), Valproic Acid (VPA), romidepsin and MS-275. In one embodiment, the HDAC inhibitor is romidepsin.

Alkylating agents useful in the methods provided herein include, but are not limited to, uramustine, chlorambucil, bendamustine, lomustine, streptozocin, busulfan, nedaplatin, oxaliplatin, satraplatin, and triplatin tetranitrate. In one embodiment, the alkylating agent is bendamustine.

The hematological malignancies treated by the methods provided herein include, but are not limited to, lymphomas, leukemias, multiple myeloma, plasma cell-derived cancers, relapsed hematological malignancies, and refractory hematological malignancies. In one embodiment, lymphomas that can be treated by the methods provided herein include, but are not limited to, non-Hodgkin's lymphomas, mature B-cell lymphomas, mature T-cell and natural killer cell lymphomas, Hodgkin's lymphomas and immunodeficiency-associated lymphoproliferative disorders. In another embodiment, lymphomas that can be treated by the methods provided herein include, but are not limited to, small lymphocytic lymphoma, follicular lymphoma, Mantle cell lymphoma, diffuse large B-cell lymphoma, Burkitt lymphoma, B-cell lymphoblastic lymphoma, small cleaved B-cell lymphoma, non-cleaved B-cell lymphoma, cutaneous T-cell lymphoma (CTCL), and peripheral T-cell lymphoma (PTCL).

In one embodiment, lymphoma is T-cell lymphoma. In one embodiment, T-cell lymphoma is peripheral T-cell lymphoma (PTCL).

In another embodiment, provided herein is a pharmaceutical composition for treating, preventing or managing a hematological malignancy in a patient comprising an HDAC inhibitor and an alkylating agent. In one embodiment, the HDAC inhibitor is romidepsin. In another embodiment, the alkylating agent is bendamustine.

In yet another embodiment, provided herein are single unit dosage forms, dosing regimens and kits which comprise an HDAC inhibitor and an alkylating agent. In one embodiment, the HDAC inhibitor is romidepsin. In another embodiment, the alkylating agent is bendamustine.

### BRIEF DESCRIPTION OF THE DRAWINGS

FIGS. 1A and 1B depict the dose effect growth inhibition curves for romidepsin (FIG. 1A) and bendamustine (FIG. 1B) in various T-Cell lymphoma and leukemia cell lines. The “X” axis represents the logarithmic concentration of each drug, and the “Y” axis represents the normalized percentage of viable cells. The dashed line represents 50% of viable cells and the intersection with each curve is the concentration that inhibits viability by 50% (IC₅₀) in nM.

FIG. 2 depicts the effect of romidepsin (in a dose of 0.5 mg/kg) and bendamustine (in a dose of 20 mg/kg), administered alone or in combination, on the number of circulating tumor cells detected by flow cytometry in the peripheral blood of the Itk-Syk transgenic mice.

### DETAILED DESCRIPTION

### Definitions

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. It should also be noted that use of “or” means “and/or” unless stated otherwise. Furthermore, use of the term “including” as well as other forms, such as “include,” “includes,” and “included” is not limiting.

The term “treating” as used herein, means an alleviation, in whole or in part, of symptoms associated with a disorder or disease (e.g., cancer or a tumor syndrome, for example, a hematological malignancy), or slowing, or halting of further progression or worsening of those symptoms.

The term “preventing” as used herein, means the prevention of the onset, recurrence or spread, in whole or in part, of the disease or disorder (e.g., cancer, for example, a hematological malignancy), or a symptom thereof.

The term “effective amount” in connection with the HDAC inhibitor means an amount capable of alleviating, in whole or in part, symptoms associated with a disorder, for example cancer, or slowing or halting further progression or worsening of those symptoms, or preventing or providing prophylaxis for cancer, in a subject at risk for cancer. The effective amount of the HDAC inhibitor, for example in a pharmaceutical composition, may be at a level that will exercise the desired effect. As will be apparent to those skilled in the art, it is to be expected that the effective amount of an HDAC inhibitor disclosed herein may vary depending on the severity of the indication being treated.

The term “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier must be “acceptable” in the sense of being compatible with the other ingredients of the formulation and not injurious to a subject to whom it is administered. Nor should an acceptable carrier alter the specific activity of the subject compounds.

The term “pharmaceutically acceptable” refers to molecular entities and compositions that are physiologically tolerable and do not typically produce an allergic or similar untoward reaction, such as gastric upset, dizziness and the like, when administered to a human.

The term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

The term “prodrug” means a derivative of a compound that can hydrolyze, oxidize, or otherwise react under biological conditions (in vitro or in viva) to provide the compound. Examples of prodrugs include, but are not limited to, derivatives of HDAC inhibitors used in the methods described herein, that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described in Burger's Medicinal Chemistry and Drug Discovery, 172-178, 949-982 (Manfred E. Wolff, ed., 5th ed. 1995), and Design of Prodrugs (H. Bundgaard, ed., Elsevier, New York 1985).

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

The term “unit-dosage form” refers to a physically discrete unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. A unit-dosage form may be administered in fractions or multiples thereof. Examples of a unit-dosage form include an ampoule, syringe, and individually packaged tablet and capsule.

The term “multiple-dosage form” is a plurality of identical unit-dosage forms packaged in a single container to be administered in segregated unit-dosage form. Examples of a multiple-dosage form include a vial, bottle of tablets or capsules, or bottle of pints or gallons.

The term “tumor” refers to all neoplastic cell growth and proliferation, whether malignant or benign, and all pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “cancer” includes, but is not limited to, solid tumors and blood borne tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. In one embodiment, the cancer is a hematological malignancy.

The term “proliferative disorder or disease” refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders.

The term “relapsed” refers to a situation where a subject, that has had a remission of cancer after a therapy, has a return of cancer cells.

The term “refractory” or “resistant” refers to a circumstance where a subject, even after intensive treatment, has residual cancer cells in the body.

The term “lymphoma” means a type of cancer in the lymphatic cells of the immune system and includes, but is not limited to, non-Hodgkin's lymphomas, mature B-cell lymphomas, mature T-cell and natural killer cell lymphomas, Hodgkin's lymphomas and immunodeficiency-associated lymphoproliferative disorders.

The term “peripheral T-Cell Lymphoma (CTCL)” refers to a group or T-cell lymphomas that develop outside of the thymus, i.e. in lymphocytes mature white blood cells (T cell) and natural killer (NK) cells).

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The terms “co-administration” and “in combination with” include the administration of two or more therapeutic agents simultaneously, concurrently or sequentially within no specific time limits unless otherwise indicated. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., 5 minutes, 15 minutes, 30 minutes, 45 minutes, 1 hour, 2 hours, 4 hours, 6 hours, 12 hours, 24 hours. 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes. 45 minutes. 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The terms “composition,” “formulation,” and “dosage form” are intended to encompass products comprising the specified ingredient(s) (in the specified amounts, if indicated), as well as any product(s) which result, directly or indirectly, from combination of the specified ingredient(s) in the specified amount(s).

The term “alkylating agent” refers to a class of chemotherapy drugs that bind to DNA through chemical (alkyl) groups that form permanent covalent bonds with nucleophilic substances in the DNA, and prevent proper DNA replication.

The term “hydrate” means a compound provided herein or a salt thereof, which further includes a stoichiometric or non-stoichiometric amount of water bound by non-covalent intermolecular forces.

The term “solvate” means a solvate formed from the association of one or more solvent molecules to a compound provided herein. The term “solvate” includes hydrates (e.g., hemihydrate, monohydrate, dihydrate, trihydrate, tetrahydrate, and the like).

As used herein, and unless otherwise specified, the compounds described herein are intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

In one embodiment, a compound described herein is intended to encompass isotopically enriched analogs. For example, one or more hydrogen position(s) in a compound may be enriched with deuterium and/or tritium. Other suitable isotopes that may be enriched at particular positions of a compound include, but are not limited, C-13, C-14, N-15, O-17, and/or O-18. In one embodiment, a compound described herein may be enriched at more than one position with isotopes, that are the same or different.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Romidepsin

Romidepsin is a natural product which was isolated from Chromobacterium violaceum by Fujisawa Pharmaceuticals (Published Japanese Patent Application No. 64872, U.S. Pat. No. 4,977,138, issued Dec. 11, 1990, Ueda et al., J. Antibiot (Tokyo) 47:301-310, 1994; Nakajima et al., Exp Cell Res 241:126-133, 1998; and WO 02/20817; each of which is incorporated herein by reference. It is a bicyclic peptide consisting of four amino acid residues (D-valine, D-cysteine, dehydrobutyrine, and L-valine) and a novel acid (3-hydroxy-7-mercapto-4-heptenoic acid) containing both amide and ester bonds. In addition to the production from C. violaceum using fermentation, romidepsin can also be prepared by synthetic or semi-synthetic means. The total synthesis of romidepsin reported by Kahn et al. involves 14 steps and yields romidepsin in 18% overall yield (Kahn et al. J. Am. Chem. Soc. 118:7237-7238, 1996).

The chemical name of romidepsin is (1S,4S,7Z,10S,16E,21R)-7-ethyl idene-4,21-bis(1-methylethyl)-2-oxa-12,13-dithia-5,8,20,23-tetrazabicyclo[8.7.6]tricos-16-ene-3,6,9,19,22-pentone. The empirical formula is C₂₄H₃₆N₄O₆S₂. The molecular weight is 540.71. At room temperature, romidepsin is a white powder.

The structure of romidepsin is shown below (formula I):

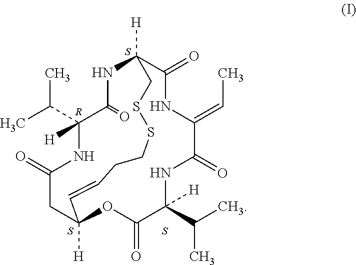

Romidepsin has been shown to have anti-microbial, immunosuppressive, and anti-tumor activities. It was tested, for example, for use in treating patients with hematological malignancies (e.g, cutaneous T-cell lymphoma (CTCL), peripheral T-cell lymphoma (PTCL), multiple myeloma, etc.) and solid tumors (e.g., prostate cancer, pancreatic cancer, etc.) and is thought to act by selectively inhibiting deacetylases (e.g., histone deacetylase, tubulin deacetylase), thus promising new targets for the development of a new class of anti-cancer therapies (Nakajima et al., Exp Cell Res 241:126-133, 1998). One mode of action of romidepsin involves the inhibition of one or more classes of histone deacetylases (HDAC). Preparations and purification of romidepsin is described, for example, in U.S. Pat. No. 4,977,138 and International PCT Application Publication WO 02/20817, each of which is incorporated herein by reference.

Exemplary forms of romidepsin include, but are not limited to, salts, esters, pro-drugs, isomers, stereoisomers (e.g., enantiomers, diastereomers), tautomers, protected forms, reduced forms, oxidized forms, derivatives, and combinations thereof, with the desired activity (e.g., deacetylase inhibitory activity, aggressive inhibition, cytotoxicity). In certain embodiments, romidepsin is a pharmaceutical grade material and meets the standards of the U.S. Pharmacopoeia, Japanese Pharmacopoeia, or European Pharmacopoeia. In certain embodiments, the romidepsin is at least 95%, at least 98%, at least 99%, at least 99.9%, or at least 99.95% pure. In certain embodiments, the romidepsin is at least 95%, at least 98%, at least 99%, at least 99.9%, or at least 99.95% monomeric. In certain embodiments, no impurities are detectable in the romidepsin materials (e.g., oxidized material, reduced material, dimerized or oligomerized material, side products, etc.). Romidepsin typically includes less than 1.0%, less than 0.5%, less than 0.2%, or less than 0.1% of total other unknowns. The purity of romidepsin may be assessed by appearance, HPLC, specific rotation, NMR spectroscopy, IR spectroscopy. UV/Visible spectroscopy, powder x-ray diffraction (XRPD) analysis, elemental analysis, LC-mass spectroscopy, or mass spectroscopy.

Romidepsin is sold under the tradename Istodax® and is approved in the United States for the treatment of cutaneous T-cell lymphoma (CTCL) in patients who have received at least one prior systemic therapy, and for the treatment of peripheral T-cell lymphoma (PTCL) in patients who have received at least one prior therapy.

Alkylating Agents

Cancer cells proliferate faster and with less error-correcting than healthy cells, and therefore are more sensitive to DNA damage, i.e., being alkylated. For this reason, alkylating agents are widely used to treat various cancers.

There are different classes of alkylating agents: classical and non classical. Classical alkylating agents include true alkyl groups, they destroy proliferating cancer cells by adding an alkyl group to DNA molecule and preventing its replication. The alkyl group attaches to the guanine base of DNA, at the number 7 nitrogen atom of the purine ring.

In one embodiment, classical alkylating agents suitable for use in the methods provided herein include, but are not limited to, nitrogen mustards, nitrosoureas, alkyl sulfonates, and alkylating-like agents.

In one embodiment, nitrogene mustards suitable for use in the methods provided herein include, but are not limited to, uramustine, chlorambucil, and bendamustine. In one embodiment, nitrosoureas suitable for use in the methods provided herein include, but are not limited to, streptozocin and lomustine. In one embodiment, alkyl sulfonates suitable for use in the methods provided herein include, but are not limited to, busulfan. In one embodiment, alkylating-like agents suitable for use in the methods provided herein include, but are not limited to, oxaliplatin, nedaplatin, satraplatin, and triplatin tetranitrate. In one embodiment, the alkylating agent is nitrogen mustard. In one embodiment, the nitrogen mustard is bendamustine.

In one embodiment, non classical alkylating agents suitable for use in the methods provided herein include, but are not limited to, procarbazine and altretamine.

Bendamustine

Bendamustine is a nitrogen mustard having the following formula:

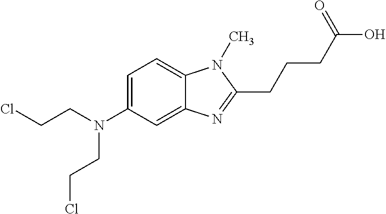 [SMILES: Cn1c(CCCC(=O)O)nc2cc(N(CCCl)CCCl)ccc21]

### 4-[5-[bis-chloroethyl)amino]-1-methylbenzimidazol-2-yl]butanoic acid

Bendamustine was first synthesized in 1963 by Ozegowski and Krebs. The compound was found to be useful for treating chronic lymphocytic leukemia, Hodgkin's disease, non-Hodgkin's lymphoma, multiple myeloma and lung cancer.

Bendamustine received its first marketing approval in Germany, where it is marketed under the tradename Ribomustin, by Astellas Pharma GmbH's licensee, Mundipharma International Corporation Limited. It is indicated as a single-agent or in combination with other anti-cancer agents for indolent non-Hodgkin's lymphoma, multiple myeloma, and chronic lymphocytic leukemia. SymBio Pharmaceuticals Ltd holds exclusive rights to develop and market bendamustine HCl in Japan and selected Asia Pacific Rim countries.

In March 2008, Cephalon received approval from the United States Food and Drug Administration (FDA) to market bendamustine in the US, where it is sold under the tradename TREANDA®, for treatment of chronic lymphocytic leukemia (Cephalon press release—Cephalon receives FDA approval for TREANDA®, a novel chemotherapy for chronic lymphocytic leukemia). In October 2008, the FDA granted further approval to market TREANDA® for the treatment of indolent B-cell non-Hodgkin's lymphoma that has progressed during or within six months of treatment with rituximab or a rituximab-containing regimen (Cephalon press release—Cephalon receives FDA approval for TREANDA® to treat patients with relapsed indolent non-Hodgkin's lymphoma).

Bendamustine is a white, water soluble microcrystalline powder with amphoteric properties. Its antiproliferative action is caused by inducing intra-strand and inter-strand cross-links between DNA bases.

After intravenous infusion it is extensively metabolized in the liver by cytochrome P450. More than 95% of the drug is bound to protein, primarily albumin. Only free bendamustine is active. Elimination is biphasic with a half-life of 6-10 minutes and a terminal half-life of approximately 30 minutes. It is eliminated primarily through the kidneys.

Bendamustine has been used both as sole therapy and in combination with other agents including etoposide, fludarabine, mitoxantrone, methotrexate, prednisone, rituximab, vincristine and ⁹⁰Y-ibritumomab tiuxetan.

The combination of bendamustine with rituximab and mitoxantrone is used for stage III/IV relapsed or refractory indolent lymphomas and mantle cell lymphoma (MCL), with or without prior rituximab-containing chemoimmunotherapy treatment (Weide et al., Leuk Lymphoma 48(7):1299-1306, 2007). The combination of bendamustine with rituximab demonstrated more than doubled disease progression-free survival in the treatment of indolent lymphoma (New combo replaces CHOP for lymphoma, December 2012 (http://www.medpageoday.com/MeetingCoverage/ASHHematology/36418). This combination also resulted in fewer side effects than the R-CHOP treatment (Rediscovered lymphoma drug helps double survival:study” (http://helath.usnews.com/health-news/news/articles/2012/06/03/rediscovered-lymphoma-drug-helps-double-survival-study). Jun. 3, 2012.

Common adverse reactions are typical for the class of nitrogen mustards, and include nausea, fatigue, vomiting, diarrhea, fever, constipation, loss of appetite, cough, headache, unintentional weight loss, difficulty breathing, rashes, and stomatitis, as well as immunosuppression, anemia, and low platelet counts.

Bendamustine has a low incidence of hair loss (alopecia) (Tageja et al., Cancer Chemother Pharmacol 66(3):413-423, 2010).

In one embodiment, bendamustine used in the methods provided herein is a free base, or a pharmaceutically acceptable salt or solvate thereof. In one embodiment, the free base or the pharmaceutically acceptable salt or solvate is a solid. In another embodiment, the free base or the pharmaceutically acceptable salt or solvate is a solid in an amorphous form. In yet another embodiment, the free base or the pharmaceutically acceptable salt or solvate is a solid in a crystalline form. For example, particular embodiments provide bendamustine in solid forms, which can be prepared using methods known in the art.

In one embodiment, bendamustine used in the methods provided herein is a pharmaceutically acceptable salt of bendamustine, which includes, but is not limited to, acetate, adipate, alginate, aspartate, benzoate, benzenesulfonate (besylate), bisulfate, butyrate, citrate, camphorate, camphorsulfonate, cyclopentanepropionate, digluconate, dodecylsulfate, 1,2-ethanedisulfonate (edisylate), ethanesulfonate (esylate), formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate (mesylate), 2-naphthalenesulfonate (napsylate), nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate, or undecanoate salts. In one embodiment, bendamustine is used in the methods provided herein as the hydrochloride salt.

Bendamustine may be synthesized by methods known in the art. In one embodiment, methods of synthesis of bendamustine include methods as disclosed in U.S. Patent Application Publication No. 2009/059765, published Apr. 5, 2010, and U.S. Patent Application Publication No 20130317234, published Nov. 28, 2013, and in the International Application Publication No. WO 2013/046223, published Apr. 4, 2013.

TREANDA® contains bendamustine hydrochloride as the active ingredient. The chemical name of bendamustine hydrochloride is 1H-benzimidazole-2-butanoic acid, 5-[bis(2-chloroethyl)amino]-1-methyl-, monohydrochloride. Its empirical molecular formula is C₁₆H₂₁C₁₂N₃O₂.HCl, and the molecular weight is 394.7.

Methods of Use

In one embodiment, provided is a method for treating, preventing, or managing a hematological malignancy in a patient, comprising administering to said patient an effective amount of an HDAC inhibitor in combination with an alkylating agent, or a pharmaceutically acceptable salt, solvate, hydrate, stereoisomer, clathrate, or prodrug thereof.

HDAC inhibitors for use in the methods provided herein include, but are not limited to, trichostatin A (TSA), Vorinostat (SAHA), Valproic Acid (VPA), romidepsin and MS-275. In one embodiment, the HDAC inhibitor is romidepsin.

Alkylating agents useful in the methods provided herein include, but are not limited to, uramustine, chlorambucil, bendamustine, lomustine, streptozocin, busulfan, nedaplatin, oxaliplatin, satraplatin, and triplatin tetranitrate. In one embodiment, the alkylating agent is bendamustine.

In one embodiment, the hematological malignancies treated by the methods provided herein include, but are not limited to, lymphomas, leukemias, multiple myeloma, plasma cell-derived cancers, relapsed hematological malignancies, and refractory hematological malignancies.

In one embodiment, the hematological malignancy is lymphoma. In one embodiment, lymphomas that can be treated by the methods provided herein include, but are not limited to, non-Hodgkin's lymphomas, mature B-cell lymphomas, mature T-cell and natural killer cell lymphomas, Hodgkin's lymphomas and immunodeficiency-associated lymphoproliferative disorders. In another embodiment, lymphomas that can be treated by the methods provided herein include, but are not limited to, small lymphocytic lymphoma, follicular lymphoma, Mantle cell lymphoma, diffuse large B-cell lymphoma, Burkitt lymphoma, B-cell lymphoblastic lymphoma, small cleaved B-cell lymphoma, non-cleaved B-cell lymphoma, cutaneous T-cell lymphoma (CTCL), and peripheral T-cell lymphoma (PTCL).

In one embodiment, the lymphoma is T-cell lymphoma. In one embodiment, the T-cell lymphoma is peripheral T-cell lymphoma (PTCL).

Administration of romidepsin and bendamustine can occur simultaneously or sequentially by the same or different routes of administration. The suitability of a particular route of administration employed for a particular active agent depends on the active agent itself and the disease being treated. In one embodiment, romidepsin and bendamustine are administered simultaneously to a subject. In one embodiment, a subject is pretreated with romidepsin before the administration of bendamustine. In one embodiment, a subject is pretreated with bendamustine before the administration of romidepsin.

Suitable routes of administration include, but are not limited to, parenteral (e.g., intraperitoneal, subcutaneous, intravenous, bolus injection, intramuscular, or intraarterial), oral, mucosal (e.g., nasal, sublingual, vaginal, buccal, or rectal), topical (e.g., eye drops or other ophthalmic preparations), transdermal or transcutaneous administration to a patient. In one embodiment, romidepsin is administered parenterally. In one embodiment, romidepsin is administered by intravenous infusion. In one embodiment, bendamustine is administered parenterally. In one embodiment, bendamustine is administered by intravenous infusion.

In one embodiment, an effective amount of romidepsin or bendamustine to be used is a therapeutically effective amount. In one embodiment, the amounts of romidepsin or bendamustine to be used in the methods provided herein include an amount sufficient to cause improvement in at least a subset of patients with respect to symptoms, overall course of disease, or other parameters known in the art. Precise amounts for therapeutically effective amounts of romidepsin or bendamustine in the pharmaceutical compositions vary depending on the age, weight, disease, and condition of a patient.

In one embodiment, romidepsin is administered intravenously, such as by intravenous infusion. In one embodiment, romidepsin is administered intravenously over a 1-6 hour period. In one embodiment, romidepsin is administered intravenously over a 3-4 hour period. In one embodiment, romidepsin is administered intravenously over a 5-6 hour period. In one embodiment, romidepsin is administered intravenously over a 4 hour period.

In one embodiment, romidepsin is administered in a dose ranging from 0.5 mg/m² to 28 mg/m². In one embodiment, romidepsin is administered in a dose ranging from 0.5 mg/m² to 5 mg/m². In one embodiment, romidepsin is administered in a dose ranging from 1 mg/m² to 25 mg/m². In one embodiment, romidepsin is administered in a dose ranging from 1 mg/m² to 20 mg/m². In one embodiment, romidepsin is administered in a dose ranging from 1 mg/m² to 15 mg/m². In one embodiment, romidepsin is administered in a dose ranging from 2 mg/m² to 15 mg/m². In one embodiment, romidepsin is administered in a dose ranging from 2 mg/m² to 12 mg/m². In one embodiment, romidepsin is administered in a dose ranging from 4 mg/m² to 12 mg/m². In one embodiment, romidepsin is administered in a dose ranging from 6 mg/m² to 12 mg/m. In one embodiment, romidepsin is administered in a dose ranging from 8 mg/m² to 12 mg/m². In one embodiment, romidepsin is administered in a dose ranging from 8 mg/m² to 10 mg/m². In one embodiment, romidepsin is administered in a dose of about 8 mg/m². In one embodiment, romidepsin is administered in a dose of about 9 mg/m². In one embodiment, romidepsin is administered in a dose of about 10 mg/m². In one embodiment, romidepsin is administered in a dose of about 11 mg/m². In one embodiment, romidepsin is administered in a dose of about 12 mg/m². In one embodiment, romidepsin is administered in a dose of about 13 mg/m². In one embodiment, romidepsin is administered in a dose of about 14 mg/m². In one embodiment, romidepsin is administered in a dose of about 15 mg/m².

In one embodiment, romidepsin is administered in a dose of 14 mg/m² over a 4 hour iv infusion on days 1, 8 and 15 of the 28 day cycle. In one embodiment, the cycle is repeated every 28 days.

In one embodiment, increasing doses of romidepsin are administered over the course of a cycle. In one embodiment, the dose of about 8 mg/m² followed by a dose of about 10 mg/m², followed by a dose of about 12 mg/m² is administered over a cycle.

In some embodiments, unit doses of romidepsin are within the range of about 0.5 mg/m² to about 28 mg/m². In certain embodiments, unit doses are in the range of about 1 mg/m² to about 25 mg/m². In certain embodiments, unit doses are in the range of about 0.5 mg/m² to about 15 mg/m². In certain embodiments, unit doses are the range of about 1 mg/m² to about 15 mg/m². In certain embodiments, unit doses are in the range of about 1 mg/m² to about 8 mg/m². In certain embodiments, unit doses are in the range of about 0.5 mg/m² to about 5 mg/m². In certain embodiments, the unit doses are in the range of about 2 mg/m² to about 10 mg/m². In some embodiments, unit doses are in the range of about 10 mg/m² to about 20 mg/m². In certain embodiments, unit doses are in the range of about 5 mg/m² to about 10 mg/m². In some embodiments, unit doses are in the range of about 10 mg/m² to about 15 mg/m². In some embodiments, unit doses are in the range of about 6 to about 19 mg/m². In some embodiments, unit doses are approximately 8 mg/m². In still other embodiments, the unit doses are approximately 9 mg/m². In still other embodiments, unit doses are approximately 10 mg/m². In still other embodiments, unit doses are approximately 11 mg/m². In still other embodiments, unit doses are approximately 12 mg/m². In still other embodiments, unit doses are approximately 13 mg/m². In still other embodiments, unit doses are approximately 14 mg/m². In still other embodiments, unit doses are approximately 15 mg/m². In still other embodiments, unit doses are approximately 30 mg/m².

In certain embodiments, different individual unit doses within the romidepsin therapy regimen are different. In some embodiments, increasing doses of romidepsin are administered over the course of a cycle. In certain embodiments, a dose of approximately 8 mg/m² is administered, followed by a dose of approximately 10 mg/m², followed by a dose of approximately 12 mg/m² may be administered over a cycle.

An amount of romidepsin administered in individual unit doses varies depending on the form of romidepsin being administered. In certain embodiments, individual unit doses of romidepsin are administered on one day followed by several days on which romidepsin is not administered. In certain embodiments, romidepsin is administered twice a week. In certain embodiments, romidepsin is administered once a week. In other embodiments, romidepsin is administered every other week.

In some embodiments, romidepsin is administered daily (for example for 2 weeks), twice weekly (for example for 4 weeks), thrice weekly (for example for 4 weeks), or on any of a variety of other intermittent schedules (e.g., on days 1, 3, and 5; on days 4 and 10; on days 1 and 15; on days 5 and 12; or on days 5, 12, and 19 of 21 or 28 day cycles).

In certain embodiments, romidepsin is administered on days 1, 8, and 15 of a 28 day cycle. In certain particular embodiments, an 8 mg/m² dose of romidepsin is administered on day 1, a 10 mg/m² dose of romidepsin is administered on day 8, and a 12 mg/m² dose of romidepsin is administered on day 15. In certain embodiments, romidepsin is administered on days 1 and 15 of a 28 day cycle with day 8 being skipped. A 28 day dosing cycle may be repeated. In certain embodiments, a 28 day cycle is repeated 2-10, 2-7, 2-5, or 3-10 times. In certain embodiments, the treatment includes 5 cycles. In certain embodiments, the treatment includes 6 cycles. In certain embodiments, the treatment includes 7 cycles. In certain embodiments, the treatment includes 8 cycles. In certain embodiments, 10 cycles are administered. In certain embodiments, greater than 10 cycles are administered.

In one embodiment, romidepsin may be formulated alone or together with one or more active agent(s), such as bendamustine, in suitable dosage unit form with pharmaceutically acceptable excipients, carriers, adjuvants and vehicles.

In one embodiment, bendamustine may be formulated alone or together with one or more active agent(s), such as romidepsin, in suitable dosage unit form with pharmaceutically acceptable excipients, carriers, adjuvants and vehicles.

In one embodiment, bendamustine is administered intravenously, such as by intravenous infusion. In one embodiment, bendamustine is administered intravenously over a 0.5-3 hour period. In one embodiment, bendamustine is administered intravenously over a 0.5-2 hour period. In one embodiment, bendamustine is administered intravenously over a 0.5-1 hour period. In one embodiment, bendamustine is administered intravenously over a 0.5 hour period. In one embodiment, bendamustine is administered intravenously over a 1 hour period.

In one embodiment, bendamustine is administered in a dose ranging from 50 mg/m² to 300 mg/m². In one embodiment, bendamustine is administered in a dose ranging from 60 mg/m² to 250 mg/m². In one embodiment, bendamustine is administered in a dose ranging from 70 mg/m² to 200 mg/m². In one embodiment, bendamustine is administered in a dose ranging from 80 mg/m² to 175 mg/m². In one embodiment, bendamustine is administered in a dose ranging from 90 mg/m² to 170 mg/m². In one embodiment, bendamustine is administered in a dose ranging from 100 mg/m² to 160 mg/m². In one embodiment, bendamustine is administered in a dose ranging from 110 mg/m² to 150 mg/m². In one embodiment, bendamustine is administered in a dose ranging from 120 mg/m² to 140 mg/m². In one embodiment, bendamustine is administered in a dose of about 40 mg/m². In one embodiment, bendamustine is administered in a dose of about 50 mg/m². In one embodiment, bendamustine is administered in a dose of about 60 mg/m². In one embodiment, bendamustine is administered in a dose of about 70 mg/m². In one embodiment, bendamustine is administered in a dose of about 80 mg/m². In one embodiment, bendamustine is administered in a dose of about 90 mg/m². In one embodiment, bendamustine is administered in a dose of about 100 mg/m². In one embodiment, bendamustine is administered in a dose of about 110 mg/m². In one embodiment, bendamustine is administered in a dose of about 120 mg/m². In one embodiment, bendamustine is administered in a dose of about 130 mg/m². In one embodiment, bendamustine is administered in a dose of about 140 mg/m². In one embodiment, bendamustine is administered in a dose of about 150 mg/m².

In one embodiment, bendamustine is administered in a dose of 100 mg/m² over 0.5 hour iv infusion on days 1 and 2 of the 28 day cycle. In one embodiment, bendamustine is administered in a dose of 120 mg/m² over 1 hour iv infusion on days 1 and 2 of the 21 day cycle. In one embodiment, bendamustine is administered in a dose of 90 mg/m² over 0.5 hour or 1 hour iv infusion on days 2 and 3 of the 28 day cycle.

In one embodiment, bendamustine is administered in an amount of from about 60 mg/m² to about 120 mg per day on days 1 and 2 of a cycle. In one embodiment, the cycle is 21 days. In one embodiment, the cycle is 28 days.

In one embodiment, bendamustine can be administered once daily or divided into multiple daily doses such as twice daily, three times daily, and four times daily. In one embodiment, the administration of the combination can be continuous (i.e., daily for consecutive days or every day), intermittent, e.g., in cycles (i.e., including days, weeks, or months of rest when no drug is administered). In one embodiment, bendamustine is administered daily, for example, once or more than once each day for a period of time. In one embodiment, bendamustine is administered intermittently, i.e., stopping and starting at either regular or irregular intervals. In one embodiment, bendamustine is administered for one to six days per week. In one embodiment, bendamustine is administered in cycles (e.g., daily administration for two to eight consecutive weeks, then a rest period with no administration for up to one week; or e.g., daily administration for one week, then a rest period with no administration for up to three weeks). In one embodiment, bendamustine is administered on alternate days. In one embodiment, bendamustine is administered in cycles (e.g., administered daily or continuously for a certain period interrupted with a rest period).

In one embodiment, bendamustine is administered once per day from one day to 14 days. In certain embodiments, bendamustine is administered once per day for 2 days. In certain embodiments, bendamustine is administered once per day for 3 days. In certain embodiments, bendamustine is administered once per day for 4 days. In certain embodiments, bendamustine is administered once per day for 5 days. In certain embodiments, bendamustine is administered once per day for 6 days. In certain embodiments, bendamustine is administered once per day for 7 days. In certain embodiments, bendamustine is administered once per day for 8 days. In certain embodiments, bendamustine is administered once per day for 9 days. In certain embodiments, bendamustine is administered once per day for 10 days. In certain embodiments, bendamustine is administered once per day for 11 days. In certain embodiments, bendamustine is administered once per day for 12 days. In certain embodiments, bendamustine is administered once per day for 13 days. In certain embodiments, bendamustine is administered once per day for 14 days.

In certain embodiments, bendamustine is administered to a patient in cycles (e.g., administration on days 1 and 4 of the 14 day cycle). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, a method provided herein comprises administering bendamustine in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or greater than about 30 cycles.

In one embodiment, bendamustine is administered to a patient at a dose provided herein over a cycle of 14 days. In one embodiment, bendamustine is administered to a patient at a dose provided herein on days 1 and 5, followed with a resting period from day 6 to day 14.

In one embodiment, a patient is administered a combination of bendamustine by iv infusion in a dose of from about 60 to about 120 mg/m² per day and romidepsin by iv infusion in a dose of from about 8 to about 14 mg/m² per day. In one embodiment, a patient is administered a combination of bendamustine by iv infusion in a dose of about 120 mg/m² per day and romidepsin by iv infusion in a dose of about 14 mg/m² per day. In one embodiment, a patient is administered a combination of bendamustine by iv infusion in a dose of about 100 mg/m² per day and romidepsin by iv infusion in a dose of about 14 mg/m² per day.

In one embodiment, one cycle comprises the iv administration of from about 60 to about 120 mg/m² per day of bendamustine on days 1 and 2 of the 28 day cycle and the iv administration of from about 8 to about 14 mg/m² per day of romidepsin on days 1, 8, and 15 of the 28 day cycle. In one embodiment, one cycle comprises the iv administration of about 100 mg/m² per day of bendamustine on days 1 and 2 of the 28 day cycle and the iv administration of about 14 mg/m² per day of romidepsin on days 1, 8, and 15 of the 28 day cycle. In one embodiment, one cycle comprises the iv administration of about 120 mg/m² per day of bendamustine on days 1 and 2 of the 21 day cycle and the iv administration of about 14 mg/m² per day of romidepsin on days 1, 8, and 15 of the 28 day cycle. In one embodiment, the number of cycles during which the combinatorial treatment is administered to a patient is to be from about 1 to about 20 cycles, or from about 1 to about 15 cycles, or from about 2 to about 8 cycles, or about 8 cycles, or about 6 cycles, or about 4 cycles.

In one embodiment, the number of cycles during which the combinatorial treatment is administered to a patient is to be from about 1 to about 20 cycles, or from about 1 to about 15 cycles, or from about 2 to about 8 cycles, or about 8 cycles, or about 6 cycles, or about 4 cycles.

In one embodiment, provided herein are methods of treating a lymphoma cell, the methods comprising treating the cell with an HDAC inhibitor and an alkylating agent. In one embodiment, the HDAC inhibitor is romidepsin. In one embodiment, the alkylating agent is bendamustine. In one embodiment, the treating is in vivo. In one embodiment, the treating is in vitro.

In one embodiment, the lymphoma is a T-cell lymphoma. In one embodiment, the T-cell lymphoma is peripheral T-cell Lymhoma (PTCL).

In one embodiment, provided herein are methods for inhibiting the growth of or killing lymphoma cells, comprising contacting the cells with an amount of romidepsin and an amount of bendamustine effective to inhibit the growth of or kill the cells.

In one embodiment, the lymphoma cells are exposed to romidepsin and bendamustine simultaneously. In one embodiment, the cells are pretreated with romidepsin before their exposure to romidepsin. In one embodiment, the cells are pretreated with bendamustine before their exposure to romidepsin.

Compositions

Romidepsin and bendamustine can be used as compositions when combined with an acceptable carrier or excipient. Such compositions are useful in the methods provided herein.

Provided herein are pharmaceutical compositions comprising romidepsin as an active ingredient, including an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug in combination with a pharmaceutically acceptable vehicle, carrier, diluent, or excipient, or a mixture thereof.

Provided herein are pharmaceutical compositions comprising bendamustine as an active ingredient or a pharmaceutically acceptable salt, solvate, hydrate, or prodrug in combination with a pharmaceutically acceptable vehicle, carrier, diluent, or excipient, or a mixture thereof.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose or other mono- or disaccharides. As used herein, the term “lactose-free” means that the amount of lactose present, if any, is insufficient to substantially increase the degradation rate of an active ingredient. In one embodiment, lactose-free compositions comprise an active ingredient provided herein, a binder/filler, and a lubricant. In another embodiment, lactose-free dosage forms comprise an active ingredient, microcrystalline cellulose, pre-gelatinized starch, and magnesium stearate.

Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, dehydrated alcohol, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

ISTODAX® (Romidepsin) Formulation

In one embodiment, romidepsin is formulated for injection as a sterile lyophilized white powder and is supplied in a single-use vial containing 10 mg romidepsin and 20 mg povidone, USP. The diluent is a sterile clear solution and is supplied in a single-use vial containing a 2 ml deliverable volume. The diluent for romidepsin contains 80% (v/v) propylene glycol, USP and 20% (v/v) dehydrated alcohol, USP. Romidepsin is supplied as a kit containing two vials.

Romidepsin for injection is intended for intravenous infusion after reconstitution with the supplied diluent and after further dilution with 0.9% Sodium Chloride, USP.

TREANDA® (Bendamustine) Formulation

TREANDA® (bendamustine hydrochloride) for Injection is intended for intravenous infusion only after reconstitution with Sterile Water for Injection, USP, and after further dilution with either 0.9% Sodium Chloride Injection, USP, or 2.5% Dextrose/0.45% Sodium Chloride Injection, USP. It is supplied as a sterile non-pyrogenic white to off-white lyophilized powder in a single-use vial. Each 25-mg vial contains 25 mg of bendamustine hydrochloride and 42.5 mg of mannitol, USP. Each 100-mg vial contains 100 mg of bendamustine hydrochloride and 170 mg of mannitol, USP. The pH of the reconstituted solution is 2.5-3.5.

Kits

In one embodiment, provided herein are kits comprising one or more containers filled with romidepsin or a pharmaceutical composition thereof, and one or more containers filled with bendamustine or a pharmaceutical composition thereof. In one embodiment, the kits additionally comprise a vial containing diluents. In one embodiment, the diluents is 80% (v/v) propylene glycol, USP and 20% (v/v) dehydrated alcohol, USP.

### Examples

### Materials and Methods

Cells and Cell Lines

Five different T-cell lymphoma and leukemia cell lines (Jurkat, HD-MAR2, Karpas 299, Sup-T1, and HH) were obtained from the American Type Culture Collection. Peripheral blood mononuclear cells (PBMC) were obtained from healthy donors. ITK-SYK-expressing cells were obtained from transgenic mice.

Assays

Cytotoxicity Assays

Cells were counted and re-suspended at an approximate concentration of 3×10⁵ per well in a 96-well plate and incubated at 37° C. in a 5% CO₂ humidified incubator for up to 72 hours. Bendamustine was added at concentrations from 1 μM to 100 μM. Romidepsin was added at concentrations from 0.1 nM to 25 nM to determine growth inhibition curves for all cell lines. In combination experiments, performed in the same conditions as for a single agent, romidepsin was added at concentrations of 2 nM to 5.5 nM, and bendamustine was added at concentrations of 5 to 20 M. These concentrations were selected to approximate the IC₅₀. After the incubation, 100 μL from each well were transferred to a 96-well opaque-walled plate; MTT assay was used to assess cell viability. Each experiment was done in triplicate and repeated at least twice.

Flow Cytometry

Jurkat, HD-MAR2, Karpas 299, Sup-T1, and HH cells were seeded at a density of 3×10⁵/mL and incubated with romidepsin (0.3-1.4 nM) and bendamustine (8-32 μM) alone or in combination for 48 or 72 hours. A minimum of 1×10⁵ events were acquired from each sample. To quantify apoptosis, the Annexin V-FITC apoptosis detection kit (Miltenyi Biotec) and propidium iodide (PI) were used according to the manufacturer's instructions. The fluorescence signals were acquired by a MACSQuant Analyzer.

Itk-Syk Transgenic Mouse Model

In vivo experiments were performed on 5- to 7-week-old NOD.CB17-Prkdcscid/J mice (Charles River Laboratories). Animals were intravenously injected with 20×10⁶ cells from an original Itk-Syk transgenic mouse (Pechloff et al, 2010). When circulating green fluorescent protein positive (GFP+) tumor cells were detected in the peripheral blood of the injected mice by flow cytometry, mice were separated into treatment groups of 9 to 10 mice each. Tumor-bearing mice were assessed for weight loss and tumor load at least twice weekly. Animals were sacrificed when tumor cells were up to 70% of total circulating peripheral blood cells or after loss of >10% body weight in accordance with institutional guidelines. Bendamustine was administered by intraperitoneal (i.p.) injection at a dose of 20 mg/kg on days 1 and 5 on a 14-day cycle. Romidepsin was administered by intraperitoneal (i.p.) injection at a dose of 2.5 mg/kg, 1.25 mg/kg and 0.5 mg/kg on days 1 and 5 on a 14-day cycle. In the combination experiments, romidepsin was administered at the dose of 0.5 mg/kg and bendamustine was administered at the dose of 20 mg/kg on days 1 and 5 on a 14-day cycle. Control groups were treated with the vehicle solution alone.

Statistical Analysis

For each cell line, the IC₅₀ was calculated with GraphPad by computing a sigmoidal dose-response curve (variable slope). The drug-drug interaction in terms of synergism, additivity, or antagonism was computed using the Chou-Talalay equation that calculates a combination index (CI): CI<1 defines synergistic effect, CI=1 defines additive effect, and CI>1 defines antagonism. For the apoptosis data, the drug-drug interactions were computed using the relative risk ratio (RRR) analysis with RRR<1 defining synergism, RRR=1 defining additivity, and RRR>1 defining antagonism. Median absolute deviation was used as a measurement of variability.

### Example 1

### Synergistic Effect of Romidepsin and Bendamustine on Cell Viability and Apoptosis in T-Cell Lymphoma Cell Lines

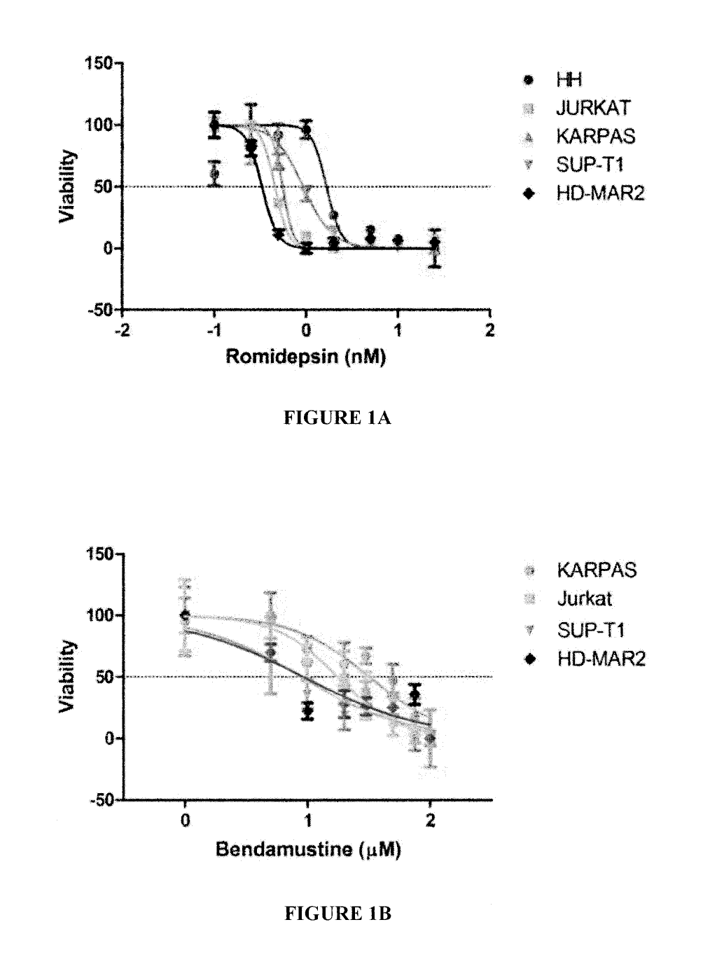

Romidepsin induced a concentration and time-dependent growth inhibition in all analysed cell lines. The IC₅₀ values for romidepsin at 48 and 72 hours were in the low nanomolar range. These values at 48 h are as follows for each cell line: Jurkat: 0.46 nM, HD-MAR2: 0.33 nM, Karpas 299: 0.56 nM, Sup-T1: 0.94 nM, and HH: 1.68 nM. The IC₅₀ values for bendamustine at 48 h were as follows: Jurkat: 17.78 M, HD-MAR2: 9.33 μM, Karpas 299: 32.04 μM, and Sup-T1: 8.88 μM. These results are shown in FIG. 1. HH cell line was resistant to bendamustine even in concentrations as high as 30 μM after 48 hours of exposure.

In combination, romidepsin (at concentrations of 2 nM to 5.5 nM) and bendamustine (at concentrations of 5 M to 20 μM) added at the concentrations selected to approximate the IC₅₀, significantly enhanced the effect of a single drug treatment reducing viability in all cell lines compared to single drug treatments. The combination showed synergism in all cell lines: Jurkat CI≦0 46; HD-MAR2 CI≦0.04; Karpas CI≦0.09; Sup-T1 CI≦0.57; and HH CI≦0 67.

Flow cytometry assays demonstrated synergy of romidepsin and bendamustine in inducing apoptosis in T-cell lymphoma cell lines. Treatment of the Karpas 299 cell line with romidepsin alone at 1 nM induced apoptosis in up to 40% of the cell population. Treatment of Karpas 299 cells with bendamustine at 10 μM (IC₅₀₋₆₀), resulted in 35% of the cells became apoptotic. The combination of romidepsin (1 nM) and bendamustine (10 μM) produced more than 70% induction of apoptosis. Treatment of SUP-T1 cells with the combination of romidepsin (10 nM) and bendamustine (50 μM) for 48 hours induced apoptosis in 74% cells, compared to 31% cells induced by romidepsin and 50% cells induced by bendamustine alone. Similar results were obtained for other analyzed cell lines.

The impact of schedule on the activity of the combination of drugs was determined by assessing cell viability after treatment with romidepsin and bendamustine as follows: (1) simultaneous exposure; (2) pretreatment with bendamustine followed by exposure to romidepsin; and (3) pretreatment with romidepsin followed by exposure to bendamustine. In all analyzed cell lines there was a significant increase in number of apoptotic cells when romidepsin was added before bendamustine. The effect was more pronounced when bendamustine was given 10 hours after romidepsin, as shown, for example, by HH cells in which apoptosis was induced in 80% of cells compared to 50% (after simultaneous exposure) or 20% (after pretreatment with bendamustine).

Primary GFP+ cells from the Itk-Syk transgenic mouse model were also exposed to romidepsin and bendamustine alone and in combination. Exposure to the combination of romidepsin (10 nM) and bendamustine (10 μM) for 48 hours induced apoptosis in 90% of the cells, compared with 60% (romidepsin) and 72% (bendamustine) of cells treated with either drug alone. The combination showed synergism with a CI of 0.08 at 48 h.

Romidepsin and bendamustine exhibited concentration- and time-dependent cytotoxicity against all tested T-cell lymphoma and leukemia cell lines and showed synergism when used in combination. Also, the combined treatment of the tested cells demonstrated induction of potent apoptosis and activation of various caspases.

### Example 2

### Activity of Romidepsin and Bendamustine Alone and in Combination in a Preclinical Model of PTCL

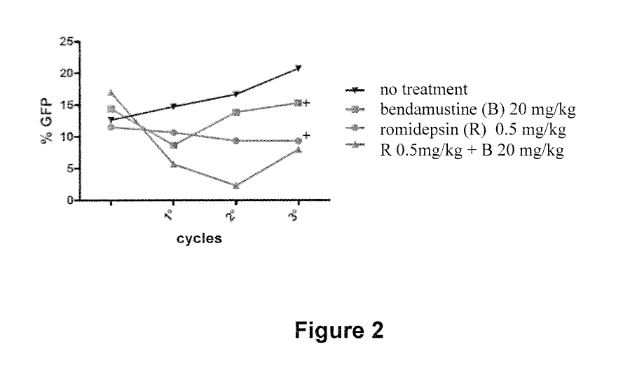

For these experiments, a mouse model of PTCL that resembles human disease was used. Mice were administered romidepsin in a dose of 0.5 mg/kg and bendamustine in a dose of 20 mg/kg on days 1 and 5 on a 14-day cycle in a combination and in a single agent experiments. The combination of the drugs was more effective in significantly reducing the number of circulating GFP+ tumor cells in the treated animals than each drug alone, as shown in FIG. 2. After the second cycle the treatment failed to control the number of circulating tumor cells that increased rapidly, as shown in FIG. 2.

These results indicate that the combination of romidepsin and bendamustine demonstrated enhanced efficacy compared with each drug when used alone.

All publications, patents, and patent applications mentioned in this specification are herein incorporated by reference to the same extent as if each individual publication, patent, or patent application was specifically and individually indicated to be incorporated by reference.

The present disclosure has been described above with reference to exemplary embodiments. However, those skilled in the art, having read this disclosure, will recognize that changes and modifications may be made to the exemplary embodiments without departing from the scope of the present disclosure. The changes or modifications are intended to be included within the scope of the present disclosure, as expressed in the following claims.

What is claimed:

1. A method of treating a T-cell lymphoma, said method comprising administering to a patient in need of such treatment a therapeutically effective amount of an HDAC inhibitor and a therapeutically effective amount of an alkylating agent.

2. The method of claim 1, wherein the T-cell lymphoma is peripheral T-cell lymphoma (PTCL).

3. The method of claim 1, wherein the HDAC inhibitor is romidepsin.

4. The method of claim 1, wherein the alkylating agent is bendamustine.

5. The method of claim 4, wherein romidepsin and bendamustine are administered simultaneously.

6. The method of claim 5, wherein romidepsin is administered prior to bendamustine.

7. The method of claim 5, wherein bendamustine is administered prior to romidepsin.

8. The method of claim 4, wherein romidepsin and bendamustine are administered by intravenous infusion.

9. The method of claim 8, wherein romidepsin is administered in an amount of from about 10 mg/m² to about 14 mg/m² and bendamustine is administered in an amount of from about 60 mg/m² to about 120 mg/m²

10. The method of claim 9, wherein romidepsin is administered in an amount of about 14 mg/m² and bendamustine is administered in an amount of about 100 mg/m.

11. The method of claim 9, wherein romidepsin is administered in an amount of about 14 mg/m² and bendamustine is administered in an amount of about 120 mg/m².

12. The method of claim 9, wherein romidepsin is administered on days 1, 8, and 15 of a 28-day cycle and bendamustine is administered on days 1 and 2 of a 28-day cycle.

13. The method of claim 9, wherein romidepsin is administered on days 1, 8, and 15 of a 28-day cycle and bendamustine is administered on days 1 and 2 of a 21-day cycle.
